(19) United States
(12) Patent Application Publication
(10) Pub. No.: US 2009/0291116 A1
(43) Pub. Date: Nov. 26, 2009

(54) Biocompatible and Biodegradable Porous Matrix in Particular Useful for Tissue Reconstruction

(75) Inventors: Daniel Casellas, Montpellier (FR); Henri Garreau, Saint Mathieu De Treviers (FR); Xavier Garric, Pavalas Les Flots (FR); Jean-Pierre Moles, Cournonsec (FR); Michel Vert, Castelnau-Le-Lez (FR)

Correspondence Address: BIRCH STEWART KOLASCH & BIRCH, PO BOX 747, FALLS CHURCH, VA, 22040-0747, US

(73) Assignees: Centre National De La Recherche Scientifique, Paris Cedex 16 (FR); Universite de Montpellier 1, Montpellier (FR)

(21) Appl. No.: 12/083,347

(22) PCT Filed: Oct. 10, 2006
(86) PCT No.: PCT/FR2006/051014

§ 371 Date: Jun. 19, 2008

(30) Foreign Application Priority Data
Oct. 11, 2005 (FR) 0553088

(51) Int. Cl. A61L 31/16; C08J 9/00; C12N 5/00; C12Q 1/02; A61K 35/12

(52) U.S. Cl. 424/426; 521/134; 435/325; 435/29; 424/93.7

(57) ABSTRACT

The invention mainly concerns a biocompatible and biodegradable porous matrix, characterized in that it is made of a three-block sequenced copolymer of formula (I): X G Y (I), wherein: G is a non-hydroxylated hydrophilic linear block, and X and Y represent respectively a hydrophobic linear polyester block. The invention further concerns the use of said matrix for coating tissue reconstruction after loss of substance or bioactive dressings.

The present invention relates to the field of reconstructing coating tissues after loss of substance or to that of bioactive dressings.

The present invention more particularly concerns a new biocompatible and biodegradable porous matrix useful for preparing scaffolds of conjunctive tissue(s), as well as said scaffolds. The invention further concerns a mesenchymatous substitute comprising said porous matrix or said scaffold of conjunctive tissue(s), associated with endothelial cells and/or fibroblasts and a coating tissue substitute, their preparation method as well as their uses.

Generally, in the field of tissue reconstruction with loss of substance, the largest advances have been made in connection with the reconstruction of bone, cartilage and coating tissue(s), more particularly including the epidermis.

The reconstruction of the epidermis is thus for example applied for treating large losses of substance as observed in badly burnt persons, and the local losses of substances as in chronic wounds and ulcers. Generally, reconstruction of integuments and notably of skin integuments, also involves application of bioactive dressings, the purpose of which is to promote healing.

As regards skin substitutes, the first of the solutions developed after the Second World War consisted in grafting heterogenic tissues. With pig xenografts and then cadaveric allografts, the patients could be kept alive in a first phase. But, confronted with the significant number of rejections and with the advent of knowledge in immunology, researchers began to turn towards the use of autologous tissues, the first technique of which was autografting. Henceforth, this technique is widely used in repair surgery services for treating skin ulcers and 2^{nd} and 3^{rd} degree burns.

Autografting consists of picking up healthy skin fragments of dermo-epidermal nature, so as to deposit them on wounds prepared by the surgeons (graft beds). This autografting technique is highly interesting because the graft-take is fast. It is also used in the form of pads for treating skin ulcers. But this autografting technique is limited because, depending on the extension of the wound, there is not always a sufficient amount of healthy skin to be picked up even if it is known how to increase in certain proportions, the surface of the autografts.

Epidermal reconstruction is a technique which was developed subsequently to autografting.

The first epidermal sheet grown in vitro was obtained by Rheinwald and Green in 1975 (Cell 6: 331-343. Serial cultivation of strains of human keratinocytes: the formation of keratinizing colonies from single cells). This type of autologous epidermal sheet (Epicel®) is used in most of the services for badly burnt persons and allows patients to be treated, for which vital prognosis is engaged. This technique consists of growing autologous keratinocytes, taken from the patient, in the presence of a nutritive layer of irradiated fibroblasts.

The drawback of this technique is localized at detachment of the sheet from its culture support. Indeed, it is necessary to use an enzymatic treatment with a proteinase, and in particular dispase, which is responsible for contraction and alteration of the sheet. These changes play a detrimental role in the graft-take.

These techniques and advances represent and will represent significant progress in therapeutics, in particular for burnt persons, but the scar obtained from epidermal sheets directly resting on the fascia is imperfect. Indeed, the epidermis is atrophic and durable fragility of the grafted skin is observed, which is therefore of insufficient quality. The lack of dermis proves to be extremely detrimental to skin reconstruction and to the mechanical properties of this new skin.

To overcome this fragility of the skin due to the lack of dermis on the graft bed, it appeared that the solution was to make an equivalent dermis in order to provide mechanical support of the epidermis while being able to limit total blood volume losses and risks of infection, after detersion of the wound. In this perspective, three equivalent dermises were developed (Integra® Dermagraft® and Transcyte®):
Integra® (Integra LifeSciences Technology). This system for covering the wound consists of two acellular layers: a first surface layer mimicking the epidermis, consists of a silicone membrane and a second deeper layer, mimicking the dermis, consists of bovine tendon collagen and of shark glycosaminoglycans. This dermal substitute forms a dermal porous matrix which is intended to be colonized by the fibroblasts of the surrounding tissues. After colonization of this matrix (21 days), the thin silicon layer is removed and then replaced with a cultivated epidermis.
Dermagraft® (Smith & Nephew). This is a vicryl resorbable net (Polyglactin 910) containing allogeneic fibroblasts. This substitute is sold frozen, which may be used on a wound at any moment. It is not rejected in spite of the presence of allogeneic fibroblasts. This equivalent dermis is particularly used in the treatment of skin ulcers. The presence of a hydrolytically degradable scaffold assumes that only the presence of water is required for its degradation and therefore this does not imply any intervention from the organism. However, this degradation may be at the origin of the release of degradation products which are more or less detrimental to keratinocyte proliferation, notably glycolic acid.
Transcyte® (Advanced Tissue Science) is a dermal substitute formed with nylon (polyamide) network on which allogeneic dermal fibroblasts were grown. This network is coated with a porcine type I collagen layer and then covered with a silicon membrane playing the role of a synthetic epidermis.

These three dermal substitutes improve the results of the graft-take and healing. It should however be noted that the presence of allogeneic fibroblasts contained in Dermagraft® and Transcyte® accelerates reappearance of a functional neodermis. It is then possible to find again mechanical properties close to the in vivo ones (owing to fast neosynthesis of elastic fibers).

These techniques therefore promote the graft-take but it is necessary to graft a culture epidermis on these equivalent dermises. As the epidermis and dermis are grown separately, the dermo-epidermal junction is lacking and is only formed during healing. A result of this is lesser resistance to removal of the epidermis and a lack of cohesion of this coating tissue substitute.

The Apligraf® product (Novartis), as for it, appears as a dermo-epidermal complex which has a bovine type I collagen dermis including allogeneic fibroblasts, an in vitro grown epidermis and a dermo-epidermal junction. This coating tissue substitute may be assimilated to skin in its composition in two layers connected through the dermo-epidermal junction but has two major drawbacks which have been the grounds for refusing it to be marketed in Europe:
the presence of foreign collagen induces a reaction of the host, the purpose of which is to destroy these collagen fibers so as to subsequently synthesize new ones which will be oriented in the tension direction of the skin,
the presence of allogeneic cells (fibroblasts and keratinocytes) which risk causing particularly for keratinocytes, a rejection reaction from the recipient organism. However, by using these allogeneic cells, the culture times may be reduced at the most and the skin substitute may be grafted on a prepared graft bed.

It is noted that, from reviewing the recalled techniques above, the proposed materials to this day may be divided in three groups: the materials of natural origin, the semi-artificial ones and the artificial ones. Materials of natural origin comprise acellular matrices such as those consisting of collagen, alginate, glycosaminoglycans, hydroxyapatite or fibrin. The artificial materials mostly include materials derived from artificial polymers such as polylactic acid, polyglycolic acid, polyethylene glycol, polyphosphazenes and many hydrogels. Semi-artificial materials combine materials of natural and artificial origin.

Finally, among the last proposed solutions applying artificial materials for reconstructing integuments, and notably skin integuments after substance loss, a dermal substitute consisting of a scaffold of polybutylene terephthalate-co-polyethylene glycol terephthalate, may be mentioned, as described in El-Ghalbzouri A, Lamme E N, van Blitterswijk C, Koopman J, Ponec M. “The use of PEGT/PBT as dermal scaffold for skin tissue engineering” Biomaterials 2004; 25(15): 2987-96.

In parallel with the field of tissue reconstruction with large substance loss, there also exists a wide field of application for tissue reconstruction with local loss of substance or healing. Moreover, most of the substitutes for coating tissue(s), as described earlier, have also been proposed as bioactive dressings intended to transiently promote healing. These healing aids are particularly important during treatment of chronic wounds such as skin ulcers.

Therefore, there is a need for finding a substitute for coating tissue(s), notably for an epidermis intended to replace autografts and allografts hitherto used in repair surgery for skin affections for which vital prognosis is engaged and which do not have the drawbacks as detailed above, this substitute may also find an application as a bioactive dressing intended to promote healing.

There further exists a need for finding a scaffold of conjunctive tissue(s) based on an artificial material with increased quality in terms of graft-take and having good biodegradability and reduced risks of immune recognition.

More specifically, the object of the present invention, is a biocompatible and biodegradable porous matrix, characterized in that it comprises a three-block sequenced copolymer fitting formula (I):

X-G-Y (I)

wherein:

G is a non-hydroxylated hydrophilic linear block, containing p recurrent units, p being a number which may vary from 150 to 700,

X and Y, either identical or different, respectively represent a hydrophobic linear polyester block, X and Y may each contain n and m recurrent units respectively, n and m each being a number which may vary from 170 to 3,500,

and in that it has pore diameters varying from 20 to 500 μm.

According to an advantageous embodiment, the porous matrix according to the invention has hydrophilicity.

The invention further concerns a method for preparing a matrix as defined above, characterized in that it comprises at least one step for preparing a copolymer as defined earlier, followed by a step for forming pores within the thereby prepared copolymer.

Moreover the object of the invention is a scaffold of conjunctive tissue(s), comprising at least one matrix as defined above, associated with at least one biocompatible and degradable filling agent.

It further concerns a mesenchymatous substitute, useful for reconstructing conjunctive tissue(s) and/or for healing, comprising a porous matrix or a scaffold of conjunctive tissues as defined earlier, associated with endothelial cells and/or fibroblasts.

Its object is also a method for preparing said mesenchymatous substitute as defined above, comprising a step for putting a porous matrix or a scaffold of conjunctive tissue(s) as described earlier, in contact with endothelial cells and/or fibroblasts and then a step for cellular proliferation.

Its object is also a substitute for coating tissue(s) characterized in that it comprises at least one porous matrix, one scaffold of conjunctive tissue(s) and/or one mesenchymatous substitute as defined earlier, associated with epithelial cells.

The object of the invention is also a method for preparing said coating tissue substitute, characterized in that it comprises a step for putting a porous matrix, a scaffold of conjunctive tissue(s) and/or a mesenchymatous substitute as defined earlier, in contact with epithelial cells, and then a cell proliferation step.

Finally, the present invention concerns the use of the porous matrix according to the invention for developing a scaffold of conjunctive tissue(s) as defined earlier or equivalent.

It also concerns the use of a porous matrix or of a scaffold of conjunctive tissue(s) according to the invention in order to obtain a mesenchymatous substitute as defined earlier or equivalent.

It also concerns the use of a mesenchymatous substitute according to the invention for obtaining a substitute for coating tissue(s) as defined earlier.

Its object is also a use of the porous matrix, of the scaffold of conjunctive tissue(s), of the mesenchymatous substitute or of the coating tissue substitute as defined above, for preparing a material intended to repair integuments, notably skin integuments, and/or to prepare a bioactive dressing intended to promote healing.

The porous matrices, scaffolds of conjunctive tissue(s), mesenchymatous substitutes and/or coating tissue substitutes according to the invention may also be used for diagnosis purposes. More specifically, the aforementioned materials according to the invention may be applied for purposes of evaluating, for example toxicity, activity, tolerance and/or impact of compounds, of pharmaceutical compositions, or even of treatment techniques intended for administration and/or for contacting a coating tissue such as skin for example.

The object of the present invention is thus also the use of a porous matrix, of a scaffold of conjunctive tissue(s), of a mesenchymatous substitute and/or of a coating tissue substitute for purposes of conducting an in vitro diagnosis test.

Within the scope of the present invention:
“block” means a portion of a macromolecule comprising several identical or different constitutive units and which has at least one structure or configuration particularity so that it may be distinguished from its adjacent portions,
“biocompatible material” means a material which is capable of fulfilling its function without any detrimental effect on the biological surroundings into which it is introduced. In the case of tissue reconstruction, its cell supporting and organ substituting function implies that biocompatibility also encompasses the notion of cytocompatibility or aptitude of the material as a culture support. The second main axis of biocompatibility is innocuity of the material towards the organism,
“biodegradable material” means a material which may be altered upon coming into contact with living cells so that its integrity is altered at a molecular level by a process related to biological activity. In particular, the notion of biodegradability implies a property loss, possibly a disappearance of the implantation site but not necessarily an elimination of the organism,
“bioresorbable material” means a material which essentially degrades and for which there is proof that the degradation products are integrated into the biomass and/or removed from the organism by metabolization or renal filtration,
“integument” means a peripheral tissue which for the body of the animal forms a continuous wall, at which a general supporting and protective role may be recognized with regards to the external medium. For example, in the particular case of skin, the term “integument” not only refers to actual epidermal tissues and formations, but also to underlying tissues included in the vague name of “dermis”,
“bioactive dressing” means a medical device which may be deposited or implanted on a tissue temporarily or definitively, for stimulating its reconstruction, said dressing according to one alternative, may be active upon coming into contact with a living organism or an extract of a living organism,
“scaffold of conjunctive tissue(s)” means an acellular material associated with at least filling agent capable of being used as a structure for a cell culture intended to form a mesenchymatous substitute,
“mesenchymatous substitute” means the material resulting from the culture of endothelial cells and/or fibroblasts upon coming into contact with a scaffold of conjunctive tissue(s), a porous matrix or equivalent,
“substitute for coating tissue(s)” means the material combining a synthetic (for example silicone) or biological (for example a coating epithelium) barrier and a mesenchymatous substitute or a scaffold of conjunctive tissue(s),
“coating epithelium” means a tissue formed with juxtaposed joined cells, integral with each other by junction systems, and separated from the adjacent tissue by a basal lamina, which covers the inside of the body and certain cavities of the organism. The cavities of the organism, more particularly relevant according to the invention, are an extension of the outside world, such as the anatomical airways, the alimentary tract, the urinary tract and the genital tract.

As a non-limiting example of relevant coating epithelia according to the invention, the epidermis, the mucosas or the exocrine gland ducts may notably be mentioned.

The coating epithelia may be monolayer (a single cell layer), laminated or pseudo-laminated.

The terms “between . . . and . . . ” and “varying from . . . to . . . ” mean that the limits are also described.

Biocompatible and Biodegradable Porous Matrix

The matrix comprises a three-block sequenced copolymer fitting formula (I):

X-G-Y (I)

wherein:

G is a non-hydroxylated hydrophilic linear block, containing p recurrent units, p being a number which may vary from 150 to 700,

X and Y, either identical or different, respectively represent a hydrophobic linear polyester block, X and Y may each contain n and m recurrent units, respectively, n and m each being a number which may vary from 170 to 3,500, in particular from 200 to 3,000, and notably from 300 to 2,500.

Copolymers related to the copolymers defined above, are known as such. They are notably known for their use in forming hydrogels as described in Patent EP 863 933 B1.

According to a preferential alternative of the invention, n and m may vary independently of each other from 200 to 3,000 and even more preferably from 300 to 2,500.

p advantageously varies from 180 to 650 and even more preferably from 200 to 600.

According to another preferential alternative of the invention, the ratio (m+n)/p is between 0.48 and 47.

Advantageously, this ratio is between 0.60 and 33 and more particularly between 1 and 25, for example between 1 and 15.

The copolymer according to the invention is advantageously hydrophilic.

The blocks which X and Y represent in formula (I), are notably aliphatic polyesters.

It is known that aliphatic polyesters may be obtained:

a) by polycondensation of a hydroxyacid on itself or by polycondensation of several hydroxyacids,

b) by polymerisation with opening of a lactone cycle,

c) or even by polycondensation of diacids and diols.

The aliphatic polyesters which may be used for synthesizing a matrix according to the invention, comprise but are not limited thereto, homopolymers and copolymers of lactide (including lactic acid, D-, L- and meso-lactide); glycolide (including glycolic acid); ε-caprolactone; p-dioxanone (1,4-dioxan-2-one); trimethylene carbonate (1,3-dioxan-2-one); alkyl derivatives of trimethylene carbonate; d-valerolactone; β-butyrolactone; γ-butyro-lactone; ε-decalactone, hydroxybutyrate; hydroxyvalerate; 1,4-dioxepan-2-one (including its 1,5,8,12-tetraoxacyclo-tetradecane-7,14-dione dimers); 1,5-dioxepan-2-one; 6,6-dimethyl-1,4-dioxan-2-one; 2,5-diketomorpholine; pivalolactone; α,α-diethylpropriolactone; ethylene carbonate; ethylene oxalate; 3-methyl-1,4-dioxane-2,5-dione; 3,3-diethyl-1,4-dioxane-2,5-dione; 6,6-dimethyl-dioxepan-2-one; 6,8-dioxabicycloctan-7-one and polymer mixtures thereof.

The aliphatic polymers used in the present invention may be (random or block, comb or alternating) homopolymers or copolymers.

More specifically, the aliphatic polyester may notably selected from (i) a homopolymer which is derived from monomers selected from lactic acid, monoesters of malic acid, lactides, glycolide, para-dioxanone, ε-caprolactone, (ii) a copolymer which is derived from the aforementioned monomers, or (iii) a polymer obtained by polycondensation of diacids and diols.

Among the polyesters derived from hydroxyacids, it is notably possible to mention those which are derived from monomers selected from lactic acid, glycolic acid, from monoesters of malic acid (for example alkyl or aralkyl monoesters), or even from monoesters resulting from the monoesterification of malic acid by a hydroxylated active ingredient, notably a hydrophobic active ingredient (see for example U.S. Pat. Nos. 4,320,753 and 4,265,247), lactides, glycolide, or para-dioxanone. The blocks represented by X and Y may also be copolymers formed by said monomers together.

Among the polymers derived from diacids and diols, poly(ethyleneglycol succinate) may be mentioned for example.

Within the scope of the present invention, the term “lactide” comprises L-lactide, D-lactide, meso-lactide and mixtures thereof.

The polymer block represented by G in formula (I) is a hydrophilic polymer which may be for example selected from the following: polyethyleneglycol, polyvinylpyrrolidone, poly(vinyl alcohol), polyoxazoline and analogous copolymers.

According to a preferential alternative of the invention, the polymer block G is a polyethyleneglycol or PEG block with formula H(OCH₂CH₂)ₚOH, wherein p varies from 2,500 to 600.

Advantageously, the copolymer has a small chain length at the G polymer block, so as to impart degradation adapted to its use as a scaffold of conjunctive tissue(s), retained for the corresponding matrix.

Within the scope of the present invention, the three-block sequenced copolymers of formula (I) may have an average number molar mass between 31,000 and 550,000, preferably between 37,500 and 475,000, and even more preferably between 45,000 and 400,000, for example between 65,000 and 400,000.

The polymerization leading to the formation of X and Y links may be carried out in the presence of the preformed G polymer and with suitably functionalized ends. For example, obtaining sequenced copolymers based on poly(hydroxyacids) and polyethyleneglycol has already been described; see notably “Block copolymers of L-lactide and poly(ethylene glycol) for biomedical applications” P. Cerrai et al., Journal of Materials Science: Materials in Medicine 5 (1994) 308-313 and document EP 295 055. The starting products are lactide on the one hand and polyethyleneglycol on the other hand. Preferably, bulk polymerization is performed in the presence of a catalyst which may be a metal, an organometallic compound or a Lewis acid. Among the catalysts used, zinc powder, calcium hydride, sodium hydride, tin octanoate, zinc lactate, etc., may be mentioned. The length of the poly(hydroxyacid) chains essentially depends on the molar ratio (lactic units)/(PEG) in the initial mixture. The length of the poly(hydroxyacid) chains also increases with the duration of the reaction which may for example range from a few minutes to a few days.

By using type G polymer blocks at the functionalized ends, for example bearing terminal hydroxyl, carboxylic acid, amino, thiol, groups, etc., it is possible to obtain sequenced copolymers fitting the formula (I) above, wherein the junction between X and G and between Y and G is made via a hydrolyzable group such as an ester, amide, urethane, thioester group, etc.

The copolymers of formula (I) may also be prepared from preformed polymer blocks, by using a type G polymer, both ends of which are suitably functionalized in a known way, and type X and Y polymers with a single functionalized end so as to be able to react with a functionalized end of G, with establishment of a covalent bond.

The matrix according to the present invention is advantageously porous. This porosity plays a dual role. Thus, it provides accommodation to cells during the phase for preparing the mensenchymatous substitute according to the present invention and then its vascularization once it is set into place on the host tissue. Moreover, it imparts mechanical properties, i.e. improved flexibility, by which good compatibility with the surrounding medium may be achieved. Moreover it is noted that the copolymer must not be altered by the reaction forming pores and finally the pores should be interconnected so as to allow cell colonization and passage of nutritive fluids.

An alternative of the method for forming pores may be used, as described in Hong-Ru Lin et al., “Preparation of macroporous biodegradable PLGA scaffolds for cell attachment with the use of mixed salts as porogen additives”, J. Biomed. Mater. Res. 2002; 63(3): 271-9. Thus, this method describes sodium chloride/ammonium hydrogencarbonate mixtures at a temperature above 60° C.

Within the scope of the present invention, a method may advantageously be used, which includes at least one step for forming pores consisting of using as a porogenic agent, particles comprising ammonium hydrogencarbonate (NH₄HCO₃), preferentially as a single constituent.

Pore generation is governed by a phenomenon which may be assimilated with a percolation phenomenon. Application of known techniques for obtaining porous matrices according to the present invention is within the skills of the skilled practitioner.

According to an advantageous embodiment of the invention, a content of such a porogenic agent is used so that the mass ratio between the porogenic agent content and the content of a copolymer of formula (I) varies from 1/1 to 50/1, preferably from 5/1 to 30/1.

In particular, when the porogenic agent is ammonium hydrogencarbonate, and the copolymer of formula (I) is a three-block polylactide-polyethyleneglycol-polylactide copolymer, and notably the copolymer as illustrated in Example 1, this mass ratio advantageously varies from 1/1 to 50/1, preferably from 5/1 from 30/1.

These particles may be completely extracted from the polymer matrix by means of a two-step washing method at 70° C. by immersion in water. The thereby obtained pores may have a diameter between 20 μm and 500 μm.

Other standard techniques for forming pores may alternatively be applied but the latter should lead to an open porosity compatible with cell invasion.

According to a preferential alternative of the invention, the diameter of the pores of the matrix varies from 20 to 500 μm, preferably from 150 to 500 μm. Quantitations of the porosity, the pore diameter and the interconnection may be obtained by examination with the preferentially environmental, scanning electron microscope (ESEM). It is also possible to determine the diameter and the number of pores by optical microscopy or even to demonstrate connectivity by having an aqueous solution pass through the dry porous matrix at a flow rate of at least 200 μL per minute per square centimetre.

Of course, the density and diameter of the pores are related to the nature of the porogenic agent used, and to the applied experimental procedure.

The biocompatibility of the porous matrix according to the present invention is available along two axes as emphasized by the definition given above.

Innocuity of the matrix and of its degradation products towards the organism is sought on the one hand. Four cumulative criteria provide a check that this first axis is observed, i.e., absence of cytotoxicity, absence of immonogenicity, absence of thrombogenicity and absence of mutagenicity. Thus, within the scope of the present invention, the porous matrix advantageously and favourably meets the whole of the criteria put forward above.

On the other hand at cell level, the matrix further advantageously has good cytocompatibity.

In particular, this cytocompatibility may be demonstrated by a study of the adhesion of constitutive cells of integuments (mainly fibroblasts and epithelial cells), a study of proliferation, a study of phenotype retention and a study of vascularization.

In addition to this biocompatibility, other properties are sought so that the matrix according to the present invention may be used for the purposes of producing scaffolds of conjunctive tissue(s). Thus, the matrix preferably has malleability and a tensile strength which are as close as possible to the mechanical features of the conjunctive tissues.

Advantageously, the matrix is biodegradable or even bioresorbable.

Finally, the matrix advantageously has the property of being sterilizable and therefore compatible with a graft under optimal aseptic conditions while not affecting the properties of the matrix.

The porous matrix, object of the invention, may as such be used as bioactive dressing.

According to a preferred embodiment of the invention, the copolymer according to the invention has an average number molar mass between 65,000 and 400,000 and has a ratio (m+n)/p between 1 and 15.

According to a most preferred embodiment, the copolymer according to the present invention has an average molar mass between 80,000 and 375,000 and has a ratio ((n+m)/p) between 1.8 and 10.9.

A porous matrix comprising a copolymer according to this particular embodiment actually has many advantages.

Thus, such a porous matrix has mechanical properties particularly suitable for applications detailed hereafter such as its use for preparing scaffolds of conjunctive tissue(s), a mesenchymatous substitute, a coating tissue substitute and/or a material for repairing integuments and/or as a bioactive dressing for promoting healing. Indeed, it then has suitable malleability and tensile strength which make them most particularly suitable for surgical manipulation. Notably such a porous matrix is suitable for suture.

Moreover, hydrolytic degradation is also suitable in the sense that the porous matrix retains adequate integrity during its application as such or as one of the forms of application listed below.

Further, such a porous matrix, taken as such or as one of the forms of application listed below, has the advantage of resistance to exudates.

Finally, this preferred embodiment is particularly advantageous concerning cell proliferation. Thus, such a porous matrix taken as such or as one of the forms of application listed below, has optimized cell proliferation thanks to the particular hydrophilic-hydrophobic balance resulting from this embodiment.

Scaffold of Conjunctive Tissue(s)

The scaffold of conjunctive tissue(s) according to the present invention is characterized in that it comprises at least one biocompatible and biodegradable porous matrix as defined earlier.

According to a particular embodiment of the invention, the scaffold may be impregnated with a biocompatible and degradable filling agent so as to promote anchoring of the cells in said scaffold. The filling agent may notably be selected from either biological materials or not, containing collagen and/or fibrin.

As a filling agent, hydrophobized hyaluronic acid, plasma rich in coagulated fibrin, preferentially autologous, collagen gel or alginate gel may be mentioned.

According to a preferential embodiment of the invention, the content of biocompatible and degradable filling agent, and more particularly of collagen, is generally less than 10%, and notably between 0.05 and 7%, or even between 0.1 and 3% by dry weight based on the total weight expressed in dry material of the scaffold of conjunctive tissue(s).

Any other compound which may be involved in tissue reconstruction, in healing, in asepsis, and in controlling pain and inflammation may also be included in the scaffold. As an example, antibiotics, silver salts, anti-fungal agents, anti-inflammatory agents and anti-tumoral agents may be mentioned.

The scaffold of conjunctive tissue(s) may have all the shapes for fitting the geometry and dimensions of the lesions of coating tissue(s) and notably of the skin to be treated.

The scaffold of conjunctive tissue(s) may moreover have a thickness varying from 500 μm to 5 mm according to the required applications.

Finally, the scaffold of conjunctive tissue(s) may comprise a porous matrix as defined earlier and another type of matrix as successive layers to the extent that the properties of the scaffold are not affected by them, notably in terms of biodegradability, biocompatibility, mechanical properties, etc.

These materials are particularly of interest for reforming integuments. Indeed, the inventors noticed that these materials were actively involved in the process of epithelial cell regeneration. For example, they have a beneficial effect on healing towards tissues which have been damaged. As such, they act as a bioactive dressing.

According to an alternative embodiment, the porous material, the scaffold of conjunctive tissue(s), the mesenchymatous substitute and/or the coating tissue substitute, may be surface-coated with an organic or inorganic film, notably of the silicon type. This thereby coated coating material may be used as such or with the removable film having been removed before use. In the case of a use as such, a surface arrangement of organic or inorganic film at the moment of the application, is preferred of course.

For example the scaffold of conjunctive tissue(s) may comprise a silicon type polymer film as a protective film.

Mesenchymatous Substitute

The object of the present invention is also a mesenchymatous substitute characterized in that it comprises a porous matrix as defined earlier or a scaffold of conjunctive tissue(s) according to the invention associated with endothelial cells and/or fibroblasts.

The mesenchymatous substitute of the present invention may be used as a bioactive dressing or as a coating tissue substitute. In this alternative, a substitute for coating tissue(s) is thus obtained directly in vivo without submitting the mesenchymatous substitute to proliferation of epithelial cells in vitro, as this is explained hereafter. In other words, the epithelial cells of the host organism proliferate in situ, thereby forming an in situ coating tissue substitute.

It may be obtained by growing endothelial cells and/or fibroblasts in contact with a porous matrix or a scaffold of conjunctive tissue(s) as defined earlier.

More particularly, when the aim is to make a mesenchymatous substitute, the pores of the matrix according to the present invention may advantageously be filled with the filling agent, as defined above, and/or with endothelial cells and/or fibroblasts, preferably with fibroblasts. The thereby obtained mesenchymatous substitute may then be directly implanted or else be subject beforehand to an in vitro cell proliferation step, in order to provide a bioactive dressing and/or a coating tissue substitute.

The fibroblast proliferation step advantageously occurs under culture conditions with a supplemented medium for a period between three and fifteen days for example.

Example 4.1 illustrates this aspect of the invention.

Coating Tissue Substitute

The coating tissue substitute according to the present invention is characterized in that it comprises at least one mesenchymatous substitute associated with epithelial cells.

The epithelial cells may be more particularly selected from epithelial cells of the gastro-intestinal system (buccal cavity, notably gingival cells, esophagus, stomach, small intestine, large intestine, and rectum), bronchi, gastro-urinary system (bladder) and of the genital system (vagina) and keratinocytes in the case of epithelial cells of the epidermis.

The substitute for coating tissue(s) of the present invention may be used as a biodegradable coating tissue substitute and/or as a bioactive dressing.

It may be obtained by growing epithelial cells such as keratinocytes in contact with a mesenchymatous substitute or a scaffold of conjunctive tissue(s) as defined earlier. The thereby obtained biodegradable coating tissue substitute may be directly implanted or else be subject to an in vitro cell proliferation step. The keratinocyte proliferation step may occur under culture conditions, in a medium with or without serum, for example for a period between three and fifteen days.

Example 5 illustrates this aspect of the invention.

The four products described earlier may be used as bioactive dressings. They may notably be deposited or implanted at all the epithelia described earlier and more particularly of the skin, but further at the gums.

The inventors have moreover demonstrated that these four products have in vivo pro-angiogenic properties.

Thus, the present invention most particularly relates to a porous matrix, a scaffold, a mesenchymatous substitute, a substitute for coating tissue(s), a material for repairing integuments and a bioactive dressing according to the present invention, provided with pro-angiogenic properties.

The affections and more particularly damages of coating tissue(s), and notably of the skin, capable of being treated by implanting each of the four products described earlier; i.e. the porous material, the scaffold of conjunctive tissue(s), the mesenchymatous substitute, the coating tissue substitute according to the present invention are nevi, skin ulcers and burns in particular.

The origin of the term “nevus” designates a malformation of coating tissue(s), and notably of the skin, clinically having the aspect of a benign tumor and corresponding to a “hamartoma”. Presently, this term is more restrictive and is used in the sense of a pigmentary nevus or a nevocellular nevus.

These tumors consist of nevus cells (or nevocytes) arranged in theques (nests). According to the localization of nevic theques in integumental planes, and notably of the skin, a distinction is made between dermal nevocellular nevus, junctional nevocellular nevus, mixed or composite nevocellular nevus and blue nevus.

Skin ulcer, as for it, is defined as a chronic loss of skin substance with a spontaneous tendency to healing. This is not a disease per se but the complication of an underlying often old or serious vascular disease which determines the prognosis and the therapeutical course. Leg ulcer, which is very frequent, is invalidating and at the origin of very many hospitalizations.

Finally, the burn is a lesion of the integumental coating, and notably of the skin, due to an energy transfer between a source of heat and the integument. The most frequent burns are thermal. They may also be of electrical, chemical or ionizing radiation origin.

The present invention is illustrated by the examples which follow, without however limiting its scope.

### EXAMPLE 1

### Preparation of a Porous Matrix According to the Present Invention

1.1 Synthesis of a PLA₅₀-PEG-PLA₅₀ Copolymer

D,L-Lactide (Sigma-Aldrich) was purified by recrystallization. A dihydroxyl PEG 20000 (Fluka) was dried in vacuo before use. The promoter used, zinc dilactate (Sigma-Aldrich) was also dried in vacuo.

The three-block copolymer (consisting of two PLA₅₀ segments and one PEG 20000 segment) was obtained by polymerization with opening of D,L-lactide in the presence of PEG 20000 and of zinc dilactate according to the following procedure.

The reagents (32.7 g D,L-lactide; 5 g PEG 20000; 7 mg zinc dilactate) are dried in vacuo and placed in a long neck flask (polymerization flask) and then connected to the assembly.

With a three-way valve, reaction mixture/vacuum or reaction mixture/argon communications may be established. The mixture is melted in vacuo. The long neck of the flask is cooled (moist paper or coolant) in order to recover the possible sublimation/distillation products by recondensation.

Degassing consists in a series of argon/vacuum purges, the mixture being alternately liquid or solid (by cooling). With this degassing, it is possible to remove any oxygen trace which is an inhibitor of the polymerization.

The flask is then sealed under dynamic vacuum with a torch. Polymerization is achieved in an oven provided with a rotary stirring system.

After polymerization (15 days), the copolymers are dissolved in acetone, and then precipitated with methanol. They are then dried in vacuo until the mass is constant.

The synthesized copolymer was characterized by infrared spectroscopy and ¹H-NMR spectroscopy. The average number molar mass is 85,000 g/mol as evaluated from proton NMR analysis.

In this example, n=451; m=451; p=454 and the ratio (m+n)/p is equal to 1.98.

In parallel with the synthesis of the copolymer, a PLA₅₀ was synthesized in order to be used as a control for the whole of the investigations. This PLA₅₀ was characterized by steric exclusion chromatography: the average number molar mass is 50,000 g/mol and the polymolecularity index (Ip) is 1.4.

1.2 Formation of Porous Matrices

The salt solubilization technique developed by Hong-Ru Lin et al., cited supra, was used for making a PLA₅₀-PEG-PLA₅₀ porous matrix.

Ammonium hydrogencarbonate (NH₄HCO₃, Sigma-Aldrich) particles were sieved (<250 μm) and then dried in vacuo. These particles were then mixed with a polymer solution according to Example 1.1, in acetone (1 mg/mL). The NH₄HCO₃/PLA₅₀-PEG-PLA₅₀ mass ratio was 9/1. The mixture was finally deposited in a glass Petri dish in air and at room temperature. After evaporation of the solvent for 24 hours and then drying in vacuo for 6 hours, the obtained film consisting of polymer and ammonium bicarbonate was detached and deposited in distilled water heated to 70° C. The presence of water at this temperature causes a reaction with ammonium bicarbonate which induces the formation of ammonia and carbon dioxide. The bubbles of carbon dioxide in formation and the disappearance of the salt then give rise to pores in the bulk of polymer.

1.1 Synthesis of Another PLA₅₀-PEG-PLA₅₀ Copolymer

According to the same synthesis method, another copolymer was synthesized having an average number molar mass of 310,000 g/mol.

In this example n=2,020, m=2,020; p=545 and (n+m)/p=8.9.

### EXAMPLE 2

### Study of Hydrolytic Degradation of the Biocompatible Matrix

2.1 Equipment and Method

*Preparation of the Degradation Buffer

The retained degradation medium is a culture medium for fibroblasts:

DMEM (Dulbecco's Modified Eagle's Medium) to which are added:
Newly-born calf serum (10%) (GIBCO-BRL), and
Penicillin (100 μg/mL), Streptomycin (100 μg/mL) and amphotericin B (1 μg/mL) (GIBCO-BRL).

In order to track the degradation of the polymer, films (W×L×H: 5 mm×5 mm×0.2 mm) obtained by solvent evaporation are used. In the case of degradation of porous copolymers, the porous parts have the same dimensions as the films, except for the thickness (0.4 mm). The pores were obtained by the technique presented above. The samples with masses close to 20-30 mg, were placed in 1.5 mL of degradation buffer.

Degradation Conditions
in vitro: the samples were placed in a cell culture incubator at 37° C. with a 5% CO₂ atmosphere saturated with water.
in vivo: the samples of porous matrix implanted in the inguinal fold of a mouse are subject after recovery to an enzyme treatment with collagenase. This treatment in presence of calcium chloride (0.1 M) at 37° C. for 24 hours provided dissociation and then solubilization of the tissues which have colonized the porous matrices.

2.2 Comparative Degradation of PLA₅₀-PEG-PLA₅₀ and of PLA₅₀

To evaluate this degradation, two types of investigation were carried out:
an in vitro study for 24 weeks in a cell culture incubator.
an in vivo study for 19 weeks after implanting supports in mice sub-cutaneously.

A. In Vitro Degradation Study

The goals of this study were the followings:
compare malleability of PLA₅₀-PEG-PLA₅₀ according to Example 1.1 relatively to that of a control PLA₅₀,
evaluate the loss of mass of PLA₅₀ and of PLA₅₀-PEG-PLA₅₀ over a period widely covering the time required for obtaining a coating tissue substitute, and
determine whether porosity has an influence on degradation kinetics.

Two criteria were retained: the macroscopic aspect and the mass loss.

For PLA₅₀ and PLA₅₀-PEG-PLA₅₀, degradation of films (thickness of 200 μm) and of porous matrices (thickness of 400 μm) was tested.

a) Macroscopic Aspect and Malleability

Before degradation, all the films and porous matrices were similar in color (white) and in flexibility. The white color appeared during sterilization.

After 3 weeks of degradation, differences in aspect appear very clearly. The PLA₅₀ films had a bumpy surface and were wound on themselves. The PLA₅₀ porous matrices as well as the films and the PLA₅₀-PEG-PLA₅₀ porous matrices remained intact on the other hand. As regards flexibility, the PLA₅₀-PEG-PLA₅₀ samples could always be handled without any apparent brittleness.

After 6 weeks of degradation, the films and the porous matrices of PLA₅₀ had retained their macroscopic aspect, but they had become very delicate to handle as they were particularly brittle. On the other hand, the films and porous matrices of PLA₅₀-PEG-PLA₅₀ had retained their macroscopic aspect without occurrence of corrugated or bumpy surface and further had large flexibility compatible with surgical manipulation.

After 12 weeks of degradation, the brittleness of the PLA₅₀ samples appeared to be enhanced with an impossibility of recoating the samples in a single piece. The PLA₅₀-PEG-PLA₅₀ films as for them were wound on themselves and had lost flexibility and mechanical properties.

After 24 weeks of degradation, all the samples proved to be impossible to recover in a single piece. No (film or matrix) sample had retained mechanical properties sufficient for being the object of a surgical manipulation.

b) Mass Loss

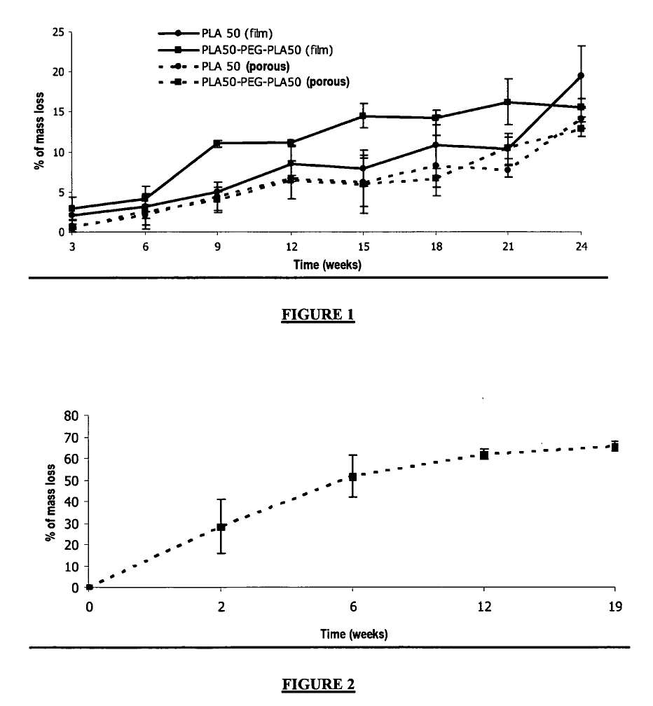

The study of degradation of PLA₅₀ (films and porous matrices) and of PLA₅₀-PEG-PLA₅₀ (films and porous matrices) was carried out in a cell culture incubator, the degradation medium being the usual culture medium for fibroblasts. The results are reported in FIG. 1, enclosed as an annex. It represents the in vitro mass loss of samples of PLA₅₀ (films and porous matrices) and of PLA₅₀-PEG-PLA₅₀ (films and porous matrices) over a period of 24 weeks.

The results correspond to the triplicate mean 6 the standard error of mean (SEM).

FIG. 1 shows that, after 6 months, the mass reductions of the samples relatively to the initial mass are 19% for the PLA₅₀ films, 16% for the PLA₅₀-PEG-PLA₅₀ films, 14% for the porous PLA₅₀ matrices and 13% for the PLA₅₀-PEG-PLA₅₀ porous matrices.

This study of degradation shows that the PLA₅₀-PEG-PLA₅₀ porous matrices are much more malleable than the PLA₅₀ porous matrices. Their mechanical properties are retained for about 12 weeks during the in vitro degradation study. The mass loss for the porous matrices does not exceed 13% after 24 weeks of degradation. Follow-up of the in vitro mass loss indicates that the porous matrices degrade more slowly than the films.

B. In Vivo Degradation Study

The object of this study was to evaluate in vivo degradation of the PLA₅₀-PEG-PLA₅₀ porous matrix according to Example 1.2. For this, the macroscopic aspect and the mass loss of samples implanted in mice subcutaneously were studied for a period of 12 weeks.

a) Macroscopic Aspect and Malleability

The implanted samples of PLA₅₀-PEG-PLA₅₀ porous matrix initially had a rectangular shape. The change in the shape of the implanted samples provides partial observation of in vivo degradation.

The samples taken after 2 weeks of implantation had retained their rectangular shape as well as their malleability.

After 6 weeks of implantation, the samples had eroded edges and were always very malleable. This appreciation of malleability was however distorted by the fact that the porous matrices had been widely colonized by the surrounding tissues, these trapped tissues in the matrix certainly strengthening the mechanical properties of the matrix.

After, 12 and 19 weeks of implantation, the samples no longer had their initial rectangular shape but an oval shape. Quite as like after 6 weeks of implantation, it was difficult to assess the malleability of the matrices. Disintegrations of the matrix were however observable particularly for the samples taken after 19 weeks.

b) Mass Loss

Degradation of the implanted samples of PLA₅₀-PEG-PLA₅₀ porous matrices was studied by tracking the mass loss and as illustrated in FIG. 2, which illustrates the in vivo mass loss of samples of PLA₅₀-PEG-PLA₅₀ porous matrices over a period of 19 weeks. The results correspond to the triplicate mean 6 the standard error of the mean (SEM). In order to only weigh the porous matrix and not the tissues which have colonized it, it was necessary to treat the samples with collagenase in order to solubilize the tissues.

According to FIG. 2, the samples have lost 26% of their initial mass after 2 weeks of implantation, 52% after 6 weeks, 62% after 12 weeks and 66% after 19 weeks.

The in vivo degradation study shows that the porous matrices rapidly degrade in vivo, the mass loss being 66% after 19 weeks. These observed differences in degradation kinetics during in vitro and in vivo studies may be explained by the presence of the cells which, through their migrations and their activities, may erode the surface of the matrix. It is also possible that permanent flow of biological fluids in vivo with respect to the relative motionlessness of the in vitro degradation media contributes to stimulating degradation of the porous matrix, as well as the dynamics related to the mobility of the animal.

### EXAMPLE 3

### Cutaneous and Gingival Cytocompatibility of the PLA₅₀-PEG-PLA₅₀ Porous Matrices

The keratinocytes are isolated from prepuces and grown in a medium without any serum. This medium allows keratinocytes to be grown in the absence of a nutritive layer (irradiated murine fibroblasts), bovine pituitary extract may replace fetal calf serum. Thus, it is possible to evaluate adhesion and specific proliferation of keratinocytes.

The serum-free medium is prepared from a volume of MCDB153 medium, to which the following supplements are added:
Bovine pituitary extract (70 μg/L) (GILCO-BRL),
Epidermal growth factor (EGF) (10 ng/mL) (GIBCO-BRL),
Penicillin (100 μg/mL), Streptomycin (100 μg/mL) and amphotericin B (1 μg/mL) (GIBCO-BRL).

Human dermal fibroblasts are isolated from prepuces. Human gingival fibroblasts are isolated from a biopsy obtained after wisdom tooth removal. Both types of fibroblasts are grown with a specific medium prepared extemporaneously: DMEM (Dulbecco's Modified Eagle's Medium) to which are added:
Newly-born calf serum (10%, by volume) (GIBCO-BRL),
Penicillin (100 μg/mL), Streptomycin (100 μg/mL) and amphoterincin B (1 μg/mL) (GIBCO-BRL).

Cell count is estimated with the MTT colorimetric test. MTT (3-(4,5-diethyl-thiazol-2-yl)-2,5-diphenyl tetrazolium bromide) is a substrate of succinate dehydrogenases which are mitochondrial enzymes. These enzymes are capable of transforming MTT into formazan crystals insoluble in an aqueous medium. The solubilization of these crystals in isopropanol and then the measurement of the optical density at 570 nm (SLT Spectra) provide evaluation of the mitochondrial activity and therefore of the cell viability and thus proportionally the number of cells.

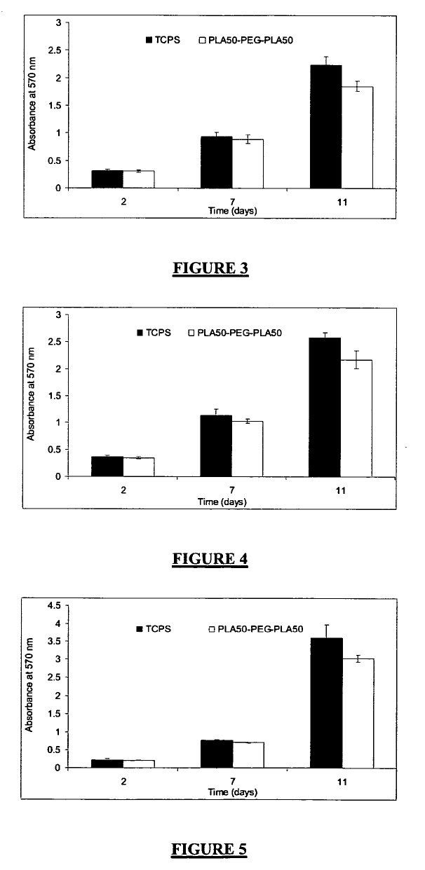

The study of fibroblast proliferation over a period of 11 days indicates that dermal and gingival fibroblasts are capable of proliferating on PLA₅₀-PEG-PLA₅₀ porous matrices as compared with polystyrene (TCPS, tissue culture polystyrene) (see FIGS. 3, 4).

FIG. 3 illustrates the study of proliferation of dermal fibroblasts on PLA₅₀-PEG-PLA₅₀ films whereas FIG. 4 illustrates the study of proliferation of gingival fibroblasts on PLA₅₀-PEG-PLA₅₀ films.

The study of keratinocyte proliferation over a period of 11 days indicates that the keratinocytes are capable of proliferating on PLA₅₀-PEG-PLA₅₀ supports, as compared with polystyrene (TCPS, tissue culture polystyrene) (see FIG. 5).

FIG. 5 illustrates the study of proliferation of keratinocytes on PLA₅₀-PEG-PLA₅₀ films.

### EXAMPLE 4

### The Use of the Biocompatible Scaffold of Conjunctive Tissue(s) in Cell Culture

4.1 For Forming a Mesenchymatous Substitute

The mesenchymatous substitute consists of a PLA₅₀-PEG-PLA₅₀ porous matrix according to Example 1.2, the pores of which are filled with rat type I collagen and human dermal fibroblasts. The type I collagen is extracted from rat tail tendons, dissolved in a 0.1% (vol./vol.) acetic acid solution. After centrifugation, the collagen is diluted in order to obtain a 3 mg/mL solution. To this solution of collagen, are added extemporaneously and chronologically:
9/1 vol./vol. DMEM/newly-born serum (culture medium for human dermal fibroblasts),
NaOH 0.1 N (50 μL/mL),
Human dermal fibroblasts (300,000 cells/mL).

Once the mixture is achieved, the latter should be immediately deposited on the porous matrix before cross-linking of collagen fibers in the pores is complete. The final amount of collagen in the mesenchymatous substitute varies in mass from 0.05 to 7% of the total mass of said substitute.

The histological analysis of the mesenchymatous substitute reveals the presence of fibroblasts in contact with the scaffold of conjunctive tissue(s).

4.2 For Vascular Colonization

The in vivo angiogenesis model used consists in implanting collagen-free porous matrices in the inguinal fold of C57/black-6 (Iffacredo) mice during a period of 45 days. The presence of collagen in vitro is mandatory in order to obtain the formation of new vessels, on the other hand in vivo, the support alone may be evaluated. Three aspects were particularly investigated:
Vascular cytocompatibility or “intimity” of the new vessels with the porous matrix,
Vascular colonization or capacity of the organism to vascularize a PLA₅₀-PEG-PLA₅₀ porous implant,
Formation of a vascular network.

Upon removing the tissue localized at the surface of the matrix, fragments of porous matrix remained attached to this tissue. The fact that the porous matrix fragments remain attached to the newly formed tissue accounts for the proximity or even the intimity of the new vessels with the surface of the porous matrix. Indeed, certain vessels surround the porous matrix fragments and vascular buds are observable.
intrajugular injection of 0.1 mg of lectin-AlexaFluor568 (isolectin IB4, Bandeiraea simplicifolia, Molecular probes) in 0.1 mL of saline, provides a demonstration of a vascular network organized in a hierarchical way.
the functionality of the vascular system developed inside the 3D network is demonstrated by the presence of smooth muscle cells specific to the arteries or arterioles characterized by expression of α-SM actin (Monoclonal Anti-alpha Smooth Muscle Actin, Mouse Ascites Fluid Clone 1A4, Sigma-Aldrich).

As a conclusion, this angiogenesis study provides the following results: 1/the porous matrix is compatible with cells of vascular origin, there even exists an “intimity” of the new vessels with the porous matrix, 2/the vascular system is capable of colonizing a PLA₅₀-PEG-PLA₅₀ porous implant. A vascular network comprising arterioles and venules is organized on and in the porous matrix.

4.3 For Epidermal Reconstruction

In order to form an in vitro laminated epidermis, a two-step culture technique was used, characterized by using a medium with serum. The first step consists of co-growing keratinocytes and murine fibroblasts in an immersed medium (a technique developed by Jim Rheinwald and Howard Green).

These murine fibroblasts (J2), the proliferation of which is inhibited by a treatment with mitomycin C (4 μg/mL) for 3 hours, play the role of nutritive cells by secreting proteins of the extra-cellular matrix which promote attachment of the keratinocytes and growth factors which stimulate proliferation of keratinocytes.

After having obtained a mono-cellular sheet of keratinocytes, the second step consists of placing the culture support for the keratinocytes on a grid which allows the epidermis to be located at the air-liquid interface. By putting it at the air-liquid interface, it is possible to mimic the physiological situation of the epidermis and thereby promote epidermal lamination.

The composition of the culture medium with serum for both steps described earlier is the following:
DMEM/HAM F12 medium (GIBCO-BRL) (2/1: vol./vol.)
Fetal calf serum (10% vol./vol.) (GIBCO-BRL)
Hydrocortisone (0.4 mg/mL) (SIGMA-ALDRICH),
Insulin (5 mg/mL) (SIGMA-ALDRICH),
Penicillin (100 μg/mL), Streptomycin (100 μg/mL) and amphotericin B (1 μg/mL) (GIBCO-BRL),
Epidermal growth factor (EGF) (10 ng/mL) (SIGMA-ALDRICH), and
Cholera toxin (0.1 nM) (SIGMA-ALDRICH).

After 20 days of growth, the samples were cut by means of a cryomicrotome. 5-15 μm cuts are fixed with cold methanol and a 4% paraformaldehyde solution. Labels by indirect immunofluorescence were obtained with specific mouse antibodies of keratin 10, keratine 6, keratine 14, involucrin, E-cadherin and integrin β1. The secondary antibody used is an anti-mouse antibody obtained in goats coupled with a derivative of fluorescein (FITC: Fluorescein Iso Thio Cyanate). Nuclear DNA was marked with a dye: Hoechst 33342 (SIGMA-ALDRICH).

Histological analysis of the substitute of coating tissue(s) reveals the presence of an epidermis including several cell layers grown on the porous matrix support as well as the presence of fibroblasts in the dermal compartment. By labeling the DNA, the morphology of the keratinocytes may be appreciated. We observed that most of the cells of the upper layers of the epidermis formed on the porous matrix include a spindle-shaped and flattened nucleus. This morphology is characteristic of keratinocytes composing the granular layer. These observations prove that keratinocytes, after having colonized the surface of the porous matrix have engaged into the terminal differentiation program thereby forming a laminated epidermis after 20 days of growth.

Characterization of this epidermis by immunohistology provides the conclusion that different specific markers of the epidermis are present.

### EXAMPLES 5

### In Vivo Integration of the Coating Tissue Substitute

After having made a skin substitute including a dermal equivalent consisting of a PLA₅₀-PEG-PLA₅₀ porous matrix as synthesized in Example 1.3, of rat type 1 collagen and of human dermal fibroblasts, and a multi-laminated human epidermis playing the role of a barrier, it appears to be essential to evaluate the possibility of integrating this coating tissue substitute in vivo.

With this perspective, samples of this type of substitute for coating tissue(s) were grafted on the back of Nude mice (immunodepressed mice for avoiding rejection of human cells) after having picked a fragment of mouse skin with dimensions corresponding to the skin substitute (W×l: 7 mm×7 mm). The graft and healing of a coating tissue substitute was followed over a period of 27 days. First of all, we observe that 4 days after the grafting, the contour of the graft is inflamed and the epidermal surface of the substitute retains the same coloration as before the grafting. 27 days after the grafting, a vascular network is organized at the periphery of the substitute and seems to colonize it. This substitute therefore appears as a good migration support for murine keratinocytes of the lips of the wound. This colonization indicates that the mouse has not rejected the coating tissue substitute.

All the examples demonstrate that the porous matrix made with PLA₅₀-PEG-PLA₅₀ is capable of forming a mesenchymatous substitute for use as a coating tissue substitute and/or bioactive dressing.

1. A biocompatible and biodegradable porous matrix, characterized in that it comprises a three-block sequenced copolymer fitting formula (I):
X-G-Y (I)
wherein:
G is a non-hydroxylated hydrophilic linear block, containing p recurrent units, p being a number which may vary from 150 to 700,
X and Y, either identical or different, respectively represent a hydrophobic linear polyester block, X and Y may each contain n and m recurrent units, respectively, n and m each being a number which may vary from 170 to 3,500,
and in that it comprises pores with a diameter varying from 20 to 500 μm.

2. The matrix according to claim 1, characterized in that n and m vary independently of each other from 200 to 3,000.

3. The matrix according to claim 1 or 2, characterized in that p varies from 180 to 650.

4. The matrix according to claim 1, characterized in that the ratio (m+n)/p is between 0.48 and 47.

5. The matrix according to claim 1, characterized in that the ratio (m+n)/p is between 0.60 and 33.

6. The matrix according to claim 1, characterized in that X and Y, either identical or different, respectively represent (i) a homopolymer which derives from monomers selected from the group consisting of lactic acid, monoesters of malic acid, lactides, glycolides, para-dioxanone, and ε-caprolactone, (ii) a copolymer which derives from the aforementioned monomers, or (iii) a polymer obtained by polycondensation of diacids and diols.

7. The matrix according to claim 1, characterized in that the block G is selected from the group consisting of poly(ethyleneglycol), poly(vinylpyrrolidone), poly(vinyl alcohol), polyoxazoline and analogous copolymers.

8. The matrix according to claim 1, characterized in that the block G is a poly(ethyleneglycol) block with formula H(OCH₂CH₂)ₚOH wherein p varies from 200 to 600.

9. A method for preparing a matrix according to claim 1, which comprises preparing a copolymer as defined in claim 1, followed by a step for forming pores within the prepared copolymer.

10. The method according to claim 9, characterized in that the pore formation step applies, as a porogenic agent, particles comprising ammonium hydrogencarbonate.

11. A scaffold of conjunctive tissue(s) characterized in that it comprises at least one matrix according to claim 1 and at least one biocompatible and degradable filling agent.

12. The scaffold according to claim 11, characterized in that the filling agent is selected from a material containing collagen and/or fibrin.

13. The scaffold according to claim 12, characterized in that the filling agent is plasma rich in coagulated fibrin.

14. The scaffold according to claim 12, characterized in that the filling agent is a collagen gel.

15. A mesenchymatous substitute useful for reconstructing conjunctive tissue(s) and/or for healing, characterized in that it comprises a porous matrix according to claim 1 or a scaffold of conjunctive tissue(s) according to claim 11, associated with endothelial cells and/or fibroblasts.

16. A method for preparing the mesenchymatous substitute useful for reconstructing conjunctive tissue(s) and/or for healing, which comprises a step for putting a porous matrix according to claim 1 or a scaffold of conjunctive tissue(s) according to claim 11, into contact with endothelial cells and/or fibroblasts, and then a cell proliferation step.

17. A coating tissue substitute, characterized in that it comprises at least one porous matrix according to any of claim 1, or at least one scaffold of conjunctive tissue(s) according to claim 1.

18. A method for preparing the coating tissue substitute which comprises a step for putting a porous matrix according to claim 1 or a scaffold of conjunctive tissue(s) according to claim 11 in contact with endothelial cells and/or fibroblasts, and then a cell proliferation step.

19.-21. (canceled)

22. A material for repairing integuments comprising the porous matrix according to claim 1.

23. A method of in vitro diagnosis of the effect of a compound or treatment technique, which comprises contacting a porous matrix according to claim 1, with the compound or treatment technique.

24. The matrix according to claim 2, characterized in that n and m vary independently of each other from 300 to 2,500.

25. The matrix according to claim 3, characterized in that p varies from 200 to 600.

26. The matrix according to claim 5, characterized in that the ratio (m+n)/p is between 1 and 25.

27. A material for preparing a bioactive dressing, which promotes healing, comprising the porous matrix according to claim 1.
